(19) United States
(12) Patent Application Publication
(10) Pub. No.: US 2024/0279877 A1
(43) Pub. Date: Aug. 22, 2024

(54) FIBROUS STERILIZABLE MATERIAL FOR PACKAGING OF MEDICAL DEVICES AND TRAY OBTAINED FROM THE MATERIAL

(71) Applicant: Ahlstrom Oyj, Helsinki (FI)

(72) Inventors: Menno Dufour, Lyon (FR); Jonathan Laloum, Paris (FR)

(21) Appl. No.: 18/570,465

(22) PCT Filed: Jun. 13, 2022
(86) PCT No.: PCT/IB2022/055470

§ 371 Date: Dec. 14, 2023

(30) Foreign Application Priority Data
Jun. 13, 2022 (FR) FR2106237

(51) Int. Cl. D21H 27/10; B65D 1/34; B65D 77/20; D21H 13/08; D21H 17/19; D21H 17/55; D21H 19/12; D21H 21/16; D21H 21/20

(52) U.S. Cl. CPC D21H 27/10; B65D 1/34; B65D 77/2024; D21H 13/08; D21H 17/19; D21H 17/55; D21H 19/12; D21H 21/16; D21H 21/20

(57) ABSTRACT

The present disclosure provides for a sterilizable fibrous material for the packaging of medical devices intended to be sterilized, characterized in that the sterilizable fibrous material is in the form of a cardboard having a basis weight of at least 180 g/m2; and the sterilizable fibrous material comprises a mixture of fibers containing at least 75% by weight of natural cellulose fibers, the length of which is less than 5 mm; and the sterilizable fibrous material has a permeability to air of at least 1.7 μm/Pas at a pressure of 1.47 kPa, measured according to the ISO 5636-3 standard. The present disclosure also provides for a sterilizable tray obtained from the sterilizable fibrous material.

### FIELD OF THE DISCLOSURE

The present disclosure relates to the field of sterilizable packages, preferably heat-sealable ones, for medical devices, in particular reusable medical devices intended to be sterilized.

The packages of the present disclosure are intended to be sterilized by suitable means, in particular and without limitation, steam, ethylene oxide, formaldehyde and/or gamma rays.

### BACKGROUND OF THE DISCLOSURE

Two types of sterilizable packaging are available for the most part, especially at hospitals.

First of all, there are boxes or rigid containers made of metal. Due to their metallic nature, they can be particularly robust. However, these boxes have the drawback of their weight and they need to be washed regularly prior to use. Thus, this leads to a substantial handling of them, not to mention that the box eventually becomes worn out and develops leaks. The containers then need to be repaired, which leads to not insignificant costs.

There has been an effort to replace these boxes with disposable flexible packages of the pouch or sheet type. It is clear that these packages are easier to use on account of their weight and their disposable nature. However, they can have inadequate mechanical strength in regard to certain medical devices being sterilized. Furthermore, they are often manufactured from non-renewable resources, which is especially the case with the majority of those manufactured from polypropylene.

Document WO 2017/168152 A1 describes a portable and disposable tray for surgical instruments. The tray is obtained by thermoforming of pulp. In practice, the instrument is stored in a sterilization bag and then positioned within the tray prior to the actual sterilization procedure. The properties of the tray make it permeable to the means of sterilization in the same way as the sterilization bag. The drawback of this system is thus the need for two elements, namely, a tray and a bag, which significantly increases the cost.

Document EP 2917408 A1 describes a cellulose-based paper with a weight between 40 and 120 g/m². The paper is reinforced on one of its surfaces and has a sealing layer on the other surface, allowing the package to be closed by means of a film of polypropylene for example, after inserting the instrument to be sterilized. Thus, in this case there is no intermediate bag. The proposed paper has the major shortcoming of being excessively pliant, making it vulnerable when the instruments being sterilized have sharp edges.

Document GB 2449418A1 describes a sheet of calendered spunbond type, composed of fibers of polypropylene, thus not being biosourced and not being biodegradable.

### SUMMARY OF THE DISCLOSURE

To the knowledge of the Applicant, no disposable packaging rigid enough to make into trays, for example, has yet been proposed in hospitals for the sterilization of medical equipment.

Consequently, a problem which the present disclosure proposes to solve is to provide a rigid disposable packaging which is resistant to tearing, in particular, and which is ideally obtained from renewable and/or compostable components.

More precisely, an objective of the present disclosure is to provide a rigid disposable material which enables the sterilizing of instruments which are intended to be sterilized without having a risk of infection with microorganisms. Thus, it needs to be both permeable to the sterilization agent, which is in practice, for example, steam, ethylene oxide, formaldehyde, and gamma rays, and a barrier to bacteria.

Another objective of the present disclosure is to provide a rigid disposable material which complies with the standard ISO 11607-1.

To accomplish this, the Applicant has succeeded in producing a cardboard whose characteristics allow it to achieve at least partially the objectives of the aforementioned standard and whose weight allows it to be transformed into trays or any other rigid receptacle.

More precisely, the present disclosure relates to a sterilizable fibrous material for the packaging of medical devices intended to be sterilized. The sterilizable fibrous material is characterized in that: it is in the form of a cardboard having a basis weight of at least 180 g/m², advantageously at least 200 g/m²; and it comprises a mixture of fibers containing at least 75% by weight of natural cellulose fibers, the length of which is less than 5 mm; and it has a permeability to air of at least 1.7, preferably at least 3.4, advantageously strictly greater than 3.4 μm/Pa·s (μm per Pascal second) at a pressure of 1.47 kPa, measured according to the ISO 5636-3 standard.

The greater the weight of the cardboard, the more rigid and easily transformable it is.

Thus, for example, the basis weight of the cardboard is advantageously at least 270 g/m², rendering it easily transformable, especially by thermoforming.

As previously mentioned, a major difficulty is to provide a sufficiently rigid material which is both permeable to the sterilization agent, such as steam, and barrier to bacteria.

In the present application, when reference is made to a standard, the applicable version is the one in force on the filing date of the priority application.

The Applicant has ascertained that particularly interesting performance is achieved in this regard when the mixture of fibers comprises: between 20% and 100% by weight of natural long cellulose fibers, the length of which is between 1 and 5 mm; and between 0% and 80% by weight of natural short cellulose fibers, the length of which is less than 1 mm.

The cellulose fibers are chosen, for example, from the group comprising pulp (short fibers and long fibers) and the fibers of annual plants, such as abaca, cotton, flax, and hemp.

Preferably, the long fibers have a length between 1 and 3 mm and preferably between 1.4 and 2.5 mm and the short fibers have a length between 0.2 and 1 mm, preferably between 0.3 and 1 mm.

In one particular embodiment, a portion of the cellulose fibers, preferably between 1 and 40% by weight, is treated with soda in order to form mercerized cellulose fibers.

The performance of the material according to the present disclosure can be further improved when the mixture contains long fibers and short fibers, the ratio of long fibers to short fibers being between 2 and 30.

In one preferred embodiment, the mixture of fibers contains between 70 and 80% of long fibers and between 15 and 25% of short cellulose fibers.

In order to increase the proportion of biosourced ingredients of the material of the present disclosure, the cellulose fibers represent at least 30% by weight, preferably at least 50% by weight, more preferably 70% by weight and even more preferably at least 90% by weight or even 95% by weight of the weight of the material.

In one particular embodiment, the mixture of fibers further comprises between at least 1% by weight, advantageously between 1 and 20% by weight, of chemical fibers.

In practice, the chemical fibers have a titer between 0.3 and 10 dtex and a length between 2.5 and 20 mm, preferably between 2.5 and 6 mm.

When present, the chemical fibers are advantageously chosen from the group comprising artificial fibers such as Lyocell and rayon, and synthetic fibers such as biopolymers like polylactic acid, polyhydroxyalcanoate, polybutylene succinates, polybutylene succinate co-adipates, polycaprolactones, polybutyrate adipate terephthalate, poly(hydroxybutyrate-co-hydroxyvalerate) or their copolymers.

The Applicant has observed that the selection of a mixture of lyocell fibers, of preferably 1.7 dtex and 6 mm and/or of rayon, in particular Danufil (1.7 dtex, 5 mm or 3 mm) made it possible to improve the permeability of the material.

Advantageously, the mixture of fibers is biosourced and/or recyclable and/or biodegradable.

Preferably, the material according to the present disclosure is biodegradable to a degree of 90%, or even 95%. Thus, the material meets the requirements of biodegradation of the standard EN 13432.

According to another characteristic and in certain embodiments, the material of the present disclosure has pores having a maximum pore diameter less than 50 μm, measured according to appendix C of standard EN 868-3. The material may also have a mean pore diameter also known as average pore diameter less than 35 μm, preferably between 10 μm and 35 μm, measured according to appendix C of standard EN 868-3. These characteristics thus allow it to comply with standard ISO 11607-1. The material can likewise comply with the standards of series EN 868. It is noted that when tested in accordance with Annex C of standard EN 868-3, the average of the pore diameters of the ten test pieces should be lower than or equal to 35 μm, and no pore diameter should be greater than 50 μm.

In practice, the material according to the present disclosure further comprises a wet strength agent, representing between 0.15 and 1% by dry weight with respect to the dry weight of cellulose, preferably on the order of 0.5%.

According to the present disclosure, the wet strength agent is chosen from the group comprising polyamine epichlorhydrin (PAE), glyoxalated resins, such as glyoxalated polyamide (GPAM), formaldehyde-based resin.

Preferably, the material according to the present disclosure further comprises a sizing agent representing preferably between 0.15 and 1% by dry weight in relation to the dry weight of cellulose, preferably on the order of 0.5%.

In practice, the sizing agent is chosen from the group comprising alkyl ketene dimer (AKD), alkenyl succinic anhydride (ASA), and rosin-based resin.

In one preferred embodiment, the material further comprises cationic starch representing less than 1% by dry weight in relation to the dry weight of cellulose, preferably on the order of 0.5%.

According to the present disclosure, the material has hydrophobic properties, preferably measured according to the standard ISO 535 using a COBB test at 60 seconds, of less than 20 g/m².

Advantageously, it complies with the standard DIN 58953-6 sections 3 and 4 in terms of bacterial barrier. The bacterial barrier properties can also be evaluated per the standard ASTM F2101. Thus, the material advantageously has a bacteria filtration efficacy (BFE) in a single layer greater than 80%, preferably greater than 95%, and more preferably greater than 99%.

As previously mentioned, it advantageously complies with the standard ISO 11607-1.

According to another characteristic, the material has a thickness of at least 200, preferably 300 μm, in order to give it optimal rigidity.

To render it heat-sealable, in particular when it is intended to be molded into a tray covered with a heat-sealable medical plastic film, said material further comprises a coating layer, the composition of which is able to render it heat-sealable.

The coating layer further allows limiting the surface roughness of the material and thus helps with the peelability of the film once it has been heat-sealed.

Advantageously, the coating contains at least one component chosen from among starch, polyvinylic alcohol (PVA), alkyl ketene dimer (AKD). The coating may further contain other components, such as bonding agents of the acrylic type, for example, or cross-linking agents, such as salts of zirconium and polyamine epichlorhydrin (PAE), for example.

In practice, the coating is applied at a rate of 4 to 30 g/m², preferably around 5 g/m².

In order to further improve the heat sealability, the material is calendered after the coating.

The present disclosure also relates to a tray or an equivalent receptacle obtained from the material described above.

The present disclosure also relates to the use of the previously described material for the fabrication of a tray.

Preferably, the tray is obtained by thermoforming.

In order to improve the thermoforming ability of the material of the present disclosure, it has a moisture level of at least 6% by weight.

In order to avoid the risks of tearing during the thermoforming, the elongation of the material in the machine direction (MD) and the cross-machine direction (CD), preferably determined according to the standard ISO 1924-2, is at least 3%.

The present disclosure also relates to a packaging for medical sterilization, comprising: a tray as previously described, or any equivalent means; and a means of tight sterilizable closure of the tray, such as a medical film of PET, PP or any other heat-sealable or gluable means.

### BRIEF DESCRIPTION OF THE DRAWINGS

FIG. 1 is a picture of a tray of the present disclosure prior to sterilization, filled with a dye mixture.

FIG. 2 is a picture of a tray of the present disclosure after sterilization, filled with a dye mixture.

FIG. 3 is a picture of a tray of the present disclosure containing metallic parts after sterilization.

FIGS. 4A to 4D are pictures of samples of the present disclosure, and comparative samples, in a home composter.

### DETAILED DESCRIPTION OF THE DISCLOSURE

The exemplary embodiments disclosed herein are illustrative of advantageous sterilizable packages, and systems of the present disclosure and methods/techniques thereof. It should be understood, however, that the disclosed embodiments are merely exemplary of the present disclosure, which may be embodied in various forms. Therefore, details disclosed herein with reference to exemplary sterilizable packages and associated processes/techniques of assembly and use are not to be interpreted as limiting, but merely as the basis for teaching one skilled in the art how to make and use the advantageous sterilizable packages and/or alternative sterilizable packages of the present disclosure.

### EXAMPLE 1: Composition of the Samples

a. Composition of the Samples of the Present Disclosure

Pour samples were fabricated, the compositions of which are given in Table 1:

TABLE 1

| | | Sample | Sample | Sample | Sample | Sample | Sample |
|---|---|---|---|---|---|---|---|
| | | 1 | 2 | 3 | 4 | 5 | 6 |
| Mixture | Sodra | 76% | 72% | 76% | 72% | 72% | 32% |
| of fibers | Blue Z | | | | | | |
| | Navia | 19% | 18% | 19% | 18% | 18% | 16% |
| | Botnia | | | | | | 32% |
| | Nordic | | | | | | |
| | Buckeye | | | | | | 20% |
| | HPZ | | | | | | |
| | Danufil | 5% | 10% | | | | |
| | 1.7 dtex - | | | | | | |
| | 5 mm | | | | | | |
| | Danufil | | | | | 10% | |
| | 1.7 dtex - | | | | | | |
| | 3 mm | | | | | | |
| | Lyocell | | | 5% | 10% | | |
| | 1.7 dtex - | | | | | | |
| | 5 mm | | | | | | |
| Wet | Kymene | 0.4 | 0.4 | 0.4 | 0.4 | 0.4 | 0.4 |
| strength | 20 Xcel | | | | | | |
| agent (in | | | | | | | |
| dry | | | | | | | |
| weight of | | | | | | | |
| cellulose) | | | | | | | |
| Starch | Hi Cat | 0.5 | 0.5 | 0.5 | 0.5 | 0.5 | 0.5 |
| | 1134A | | | | | | |
| Sizing | Fennosize | 0.5 | 0.5 | 0.5 | 0.5 | 0.5 | 0.5 |
| agent | 860D | | | | | | |
| Coating | | 4.3 with | 5.4 with | 4.5 with | 5.2 with | 7 with | 27 with |
| (g/m²)° | | 90% PVA, | 90% PVA, | 90% PVA, | 90% PVA, | 61% PVA, | 63% PVA, |
| | | 5% AKD | 5% AKD | 5% AKD | 5% AKD | 7% AKD, | 4% AKD, |
| | | and 5% | and 5% | and 5% | and 5% | 30.5 acrylic | 32% acrylic |
| | | Kymene | Kymene | Kymene | Kymene | latex, 1.5% | latex, 1% |
| | | | | | | zirconium | zirconium |
| | | | | | | salt | salt |

b. Comparison Sample

This was a spunbond composed of fibers of polypropylene and polyolefin with a basis weight of 90.5 g/m² marketed by AMCOR 095134 under the brand name ULTRA®.

### EXAMPLE 2: Characteristics of the Samples

The major characteristics of the different samples are given in Table 2.

TABLE 2

| | | EN | Sample | Sample | Sample | Sample | Sample | Sample | Comp. |
|---|---|---|---|---|---|---|---|---|---|
| Test | Unit | 868-3 | 1 | 2 | 3 | 4 | 5 | 6 | sample |
| Weight ISO | g/m² | | 297 | 275 | 278 | 293 | 284 | 332 | 90.5 |
| 536 | | | | | | | | | |
| Thickness ISO | μm | | 466 | 474 | 450 | 485 | 441 | 476 | 174 |
| 534 | | | | | | | | | |
| Permeability | ml/ | >400 ml/ | 655 | 1332 | 650 | 1104 | 2014 | 974 | 1696 |
| to air ISO | min | min at | | | | | | | |
| 5636-3 | | 1.47 kPa | | | | | | | |
| Cobb (60 s) | g/m² | <20 g/m² | 19.66 | 19.64 | 19.4 | 19.66 | 18.7 | 17 | 2.8 |
| ISO 535 | | | | | | | | | |
| Pore diameter | μm | <=35 | 12.6 | 15.9 | 14.1 | 15.2 | 14.6 | 15 | 26 |
| EN 868-3 | | (max 50) | (18) | (22.6) | (19.3) | (21.5) | (21) | (19.5) | (41) |
| Appendix C | | | | | | | | | |

Samples 1 to 5 were compliant with the standard ISO 11607-1.

### EXAMPLE 3: Characteristics Of The Sealing Of The Tray According To The Present Disclosure

a. By a Brugger Test

Sample 4 was calendered (30 kN/m at 50° C.) and subjected to a heat sealing test with a medical film using Brugger clamps at 180° C. with a force of 4 bars for 10 seconds. No tear-off of fibers was observed and the seal showed a value greater than 1.5 N/15 mm.

b. By Injection of a Dye Per Standard ASTM F 1929
i. Quality of the Seal in Absence of Sterilization

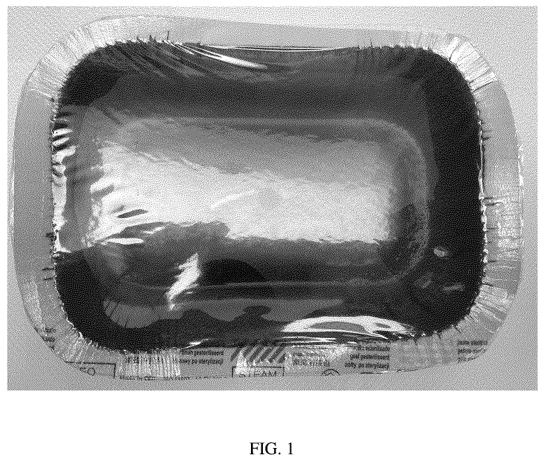

Sample 5 was thermoformed to produce a tray. The tray was sealed with a medical film for sterilization by steam or ethylene oxide made of PP/PET. An aqueous solution with a blue dye and a surfactant was injected in the tray. The colored solution was moved to the area of the sealing points on each side and maintained for 5 seconds each time, for a total duration of 20 seconds per the standard ASTM F 1929. No leak was observed (see FIG. 1). Following the standard EN 868-5, calendered sample 6 was subjected to a heat sealing test as with a medical film using Brugger clamps at 190° C. with a force of 5 bars for respectively 2, 3, 4 and 5 seconds. All samples had a peel strength greater than 1.5 N/15 mm but remained less than 5 N/15 mm. This showed that a tray according to the present disclosure can be properly sealed with a medical film and be opened without significant effort and without fibers tearing. As shown in FIG. 1, the tray extends from a first end to a second end (from left to right), and extends from a first side to a second side (from the top of FIG. 1 to the bottom of FIG. 1). The first end includes a first end wall, and the second end includes a second end wall. The first side includes a first side wall, and the second side includes a second side wall. The first and second end walls and the first and second side walls define a recess or cavity within the internal boundaries of the first and second end walls and the first and second side walls (e.g., with the recess/cavity housing the dye mixture), with a bottom wall at the bottom of the recess/cavity.

ii. Quality of the Seal after Sterilization

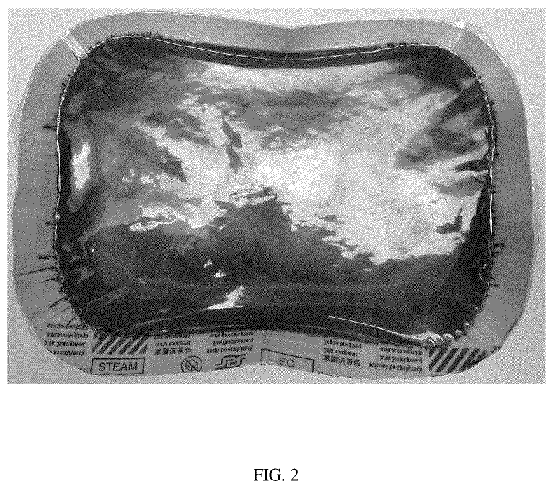

Sample 5 was thermoformed to produce a tray. The tray was then sealed with a medical film for sterilization by steam or ethylene oxide made of PP/PET, and then sterilized with steam for 18 min at 134° C. An aqueous solution with a blue dye and a surfactant was then injected in the tray. The colored solution was moved to the area of the sealing points on each side and maintained for 5 seconds each time, for a total duration of 20 seconds per the standard ASTM F 1929. No leak was observed (see FIG. 2).

### EXAMPLE 4: Test for Evaluation of Microbiological Contamination of a Tray According to the Present Disclosure

The purpose of this test was to show the ability of the material of the present disclosure to let the sterilizing steam pass through it, thus allowing a sterilization of the elements contained in it.

To do this, a tray was fabricated from sample 5. On this tray was arranged 22 metallic pieces contaminated with microorganisms contained in saliva. The tray was then heat sealed with a film for sterilization by steam or ethylene oxide made of PP/PET. The tray was then sterilized with steam at 134° C. for 18 minutes. The tray was stored under normal conditions of pressure and temperature for 15 days prior to microbial analysis of 3 metallic pieces, chosen at random from the 22 pieces.

The microbial contamination was evaluated by the method ISO 8784-1 and was based on a counting of bacteria and fungi. The medium used to culture the bacteria was Tryptic Soy Agar with an incubation of 48 h at 37° C. The medium used to culture the fungi was PDA with an incubation of 5 days at 29° C.

The results are shown in Table 3:

TABLE 3

| Sample | N of fungi | N of bacteria |
|---|---|---|
| Metallic piece 1 | <10 CFU/part | <10 CFU/part |
| Metallic piece 2 | <10 CFU/part | <10 CFU/part |
| Metallic piece 3 | <10 CFU/part | <10 CFU/part |

CFU: colony forming unit
N: total number of microorganisms calculated in UFC per piece

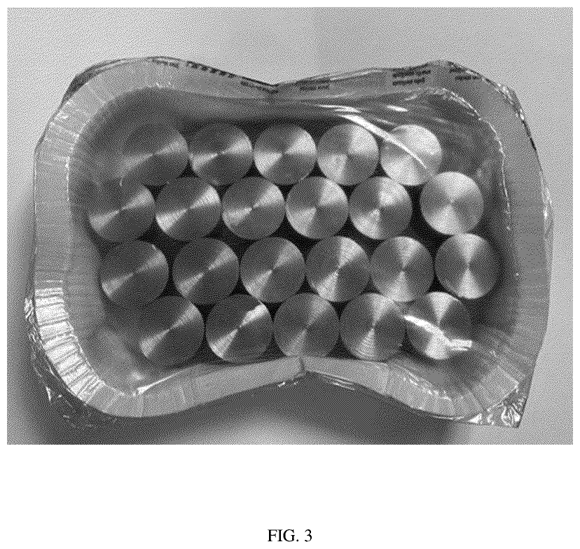

The results show that, after sterilization, the metallic pieces are no longer contaminated, which proves that the material of the present disclosure is permeable to the sterilizing steam and remains sterile after a storage at room temperature for 15 days. This test also showed the mechanical strength of the tray (see FIG. 3). In fact, it was possible to sterilize 22 metallic pieces weighing more than 1 kg and store them in sterile manner without any tearing or opening.

### EXAMPLE 5: Test for Bacterial Resistance of the Material of the Present Disclosure

a. In Humid Conditions

The test was performed on the 4 samples (samples 1-4) according to the present disclosure and one comparison sample under the conditions of the standard DIN 58953-6, section 3. Basically, the test involved sterilizing each sample with steam at 134° C. for 4 minutes. Each sample was then inoculated with 500 μL of the microbe S. epidermidis on one surface. After drying, the opposite surface of the sample was placed in contact with a culture medium and incubated for 24 h at 37° C. The two surfaces of each sample were tested.

The results are shown in Table 4.

TABLE 4
Test for bacteria resistance in humid conditions
per standard DIN 58953-6, section 3

| | | Number of CFU/agar plate | | | | | |
|---|---|---|---|---|---|---|---|
| Sample | Surface | 1 | 2 | 3 | 4 | 5 | Σ |
| Comparison sample | A | 0 | 0 | 0 | 0 | 0 | 0 |
| (Amcor ultra white | B | 0 | 0 | 0 | 0 | 0 | 0 |
| spunbond 90 g/m² | | | | | | | |
| Sample 1 | A | 0 | 0 | 0 | 0 | 0 | 0 |
| | B | 0 | 0 | 0 | 0 | 0 | 0 |
| Sample 2 | A | 0 | 0 | 0 | 0 | 0 | 0 |
| | B | 0 | 0 | 0 | 0 | 0 | 0 |
| Sample 3 | A | 0 | 0 | 0 | 0 | 0 | 0 |
| | B | 0 | 0 | 0 | 0 | 0 | 0 |
| Sample 4 | A | 0 | 0 | 0 | 0 | 0 | 0 |
| | B | 0 | 0 | 0 | 0 | 0 | 0 |
| Sample 6 | A | 0 | 0 | 0 | 0 | 0 | 0 |
| | B | 0 | 0 | 0 | 0 | 0 | 0 |

CFU = colony forming unit

No colony was found on any of the samples of the present disclosure or the comparison sample. The test for bacteria resistance in humid conditions according to standard DIN 58953-6, section 3 was thus validated.

b. In Dry Conditions
i. Standard DIN 58953-6, Section 4

The test was performed on 4 samples (samples 1-4) according to the present disclosure and one comparison sample under the conditions of the standard DIN 58953-6, section 4. Basically, the test involved sterilizing each sample with steam at 121° C. for 20 minutes. Each sample was then inoculated with 250 mg of sand contaminated with endospores of B. subtilis. Incubations were then done at 8° C. and 50° C. to create a flow of air through the contaminated sand and the sample as far as the agar plate. The samples were then incubated for 24 h at 37° C. The 2 surfaces of each sample were tested.

TABLE 5
Test for bacteria resistance in dry conditions
per standard DIN 58953-6, section 4

| | | Number of CFU/agar plate | | | | | |
|---|---|---|---|---|---|---|---|
| Sample | Surface | 1 | 2 | 3 | 4 | 5 | Σ |
| Comparison sample | A | 0 | 0 | 0 | 0 | 0 | 0 |
| (Amcor ultra white | B | 0 | 0 | 0 | 0 | 0 | 0 |
| spunbond 90 g/m² | | | | | | | |
| Sample 1 | A | 0 | 0 | 0 | 0 | 0 | 0 |
| | B | 0 | 0 | 0 | 0 | 0 | 0 |
| Sample 2 | A | 0 | 0 | 0 | 0 | 0 | 0 |
| | B | 0 | 0 | 0 | 0 | 0 | 0 |
| Sample 3 | A | 0 | 0 | 0 | 0 | 0 | 0 |
| | B | 0 | 0 | 0 | 0 | 0 | 0 |
| Sample 4 | A | 0 | 0 | 0 | 0 | 0 | 0 |
| | B | 0 | 0 | 0 | 0 | 0 | 0 |
| Sample 6 | A | 0 | 0 | 0 | 0 | 0 | 0 |
| | B | 0 | 0 | 0 | 0 | 0 | 0 |

CFU = colony forming unit

As expected, no colony was found on any of the samples of the present disclosure or the comparison sample. This was expected, inasmuch as the test in humid environment according to standard DIN 58953-6 section 3 is challenging, water serving as the vector of contamination. The test for bacteria resistance under dry conditions according to standard DIN 58953-6, section 4 was thus validated.

ii. Bacterial Filtration Efficiency (BFE)

This test was performed according to the standard ASTM F2101, using an aerosol of Staphylococcus aureus. This test expresses the ratio of the number of bacteria arrested per sample, divided by the number of bacteria sprayed onto the sample tested.

TABLE 6

| | Bacterial | | Bacterial | | Bacterial |
|---|---|---|---|---|---|
| | filtration | Comparison | filtration | Comparison | filtration |
| Sam- | efficiency | sample, 1 | efficiency | sample, 2 | efficiency |
| ple 5 | (BFE) % | sheet | (BFE) % | sheets | (BFE) % |
| 1 | 99.9 | 1 | 76.8 | 1 | 93.6 |
| 2 | 99.9 | 2 | 71.0 | 2 | 91.0 |
| 3 | 99.9 | 3 | 71.5 | 3 | 92.0 |
| 4 | 99.9 | 4 | 73.4 | 4 | 94.3 |
| 5 | 99.9 | 5 | 63.5 | 5 | 94.9 |

As shown by Table 6, sample 5 has a BFE of 99.9% as compared to the comparison sample, which has a BFE of 71.24% for a packaging of 1 sheet and 93.16% for a packaging of 2 sheets. The sample according to the present disclosure presents a more tortuous pathway for the microorganisms and thus minimizes the risks of contamination.

Breathability is the ability of a material to allow water vapor to pass through it while preventing liquid water from passing through it, and is expressed interchangeably as “water vapor transmission rate” (WVTR), or “moisture vapor transmission rate” (MVTR). Accordingly, the material of the present disclosure has an overall water vapor transmission rate (WVTR) of at least 400 g/m²/day, preferably at least 800 g/m²/day, and more preferably at least 1000 g/m²/day at 38° C. and 90% relative humidity and as determined by the ISO 2528 standard.

### Example on MVTR and Bowie and Dick Test:

The WVTR of sample 6 and comparative samples were measured according to the ISO 2528 standard, and the results are summarized in Table 7.

TABLE 7

| | WVTR |
|---|---|
| Samples | (g/m²/day) |
| Sample 6 (single layer) | 2071 |
| Sample 6 (double layer) | 1698 |
| Comparative sample | 2146 |
| (single layer) | |
| Comparative sample | 1492 |
| (single layer) | |

The samples were measured following the ISO 2528 standard and at 38° C. and 90% relative humidity. The data shows that sample 6 has comparable WVTR to the comparative sample, while the basis weight of sample 6 is more than triple of the comparative sample. The material according to the present disclosure can thus be efficiently sterilized.

In order to verify the uniformity and efficiency of the vapor penetration, sample 6 was thermoformed into a tray and a Bowie and Dick test kit was placed in the tray. The tray was then sealed with a medical film and was subjected to a sterilization cycle (Bowie and Dick cycle: 3.5 minutes at 134° C.). After sterilization, the Bowie and Dick kit test showed efficient and uniform steam penetration.

### Biodegradability and Compostability

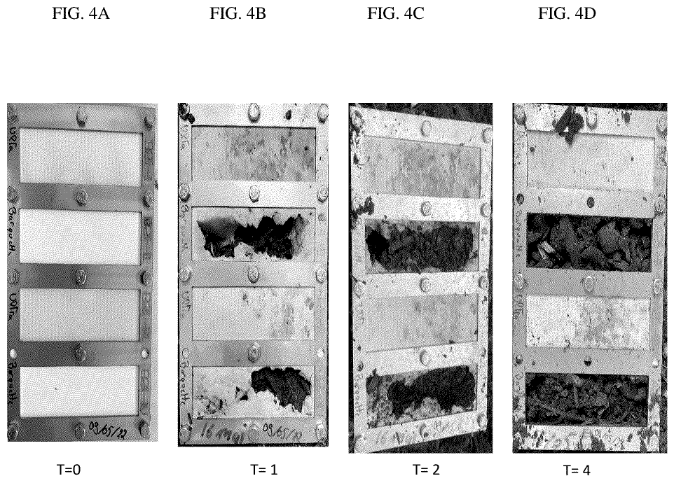

Two of each sample 6 (second from top row, and bottom row of FIGS. 4A to 4D) and comparative samples (top row, and one above the bottom row of FIGS. 4A to 4D) were placed in frame and were introduced in a home composter. The samples were then visually inspected after one, two weeks and four weeks. As it can be seen in FIGS. 4A to 4D, sample 6 has almost completely biodegraded after four weeks, whereas the comparative samples remained intact.

### Stiffness and Rigidity

In order to form a rigid tray, the material according to the present disclosure had a higher stiffness compared to other sterilization materials such as pouches or sterilization wraps. The rigidity or stiffness of a material may be measured by determining the resistance to bending of the material following ISO 2493-2-Paper and Board—Determination of resistance to bending—part 2: Taber Type tester Standard. The material according to the present disclosure may have a resistance to bending of at least 1000 mN in MD and CD directions, preferably at least 2000 mN and even more preferably at least 3000 mN.

### Example On Resistance To Bending

The resistance to bending of Sample 6 and comparative sample were measured in the machine direction (MD) and the cross-machine direction (CD) (see Table 8, below) following the ISO 2493-2 standard.

TABLE 8

| | Resistance in | Resistance in |
|---|---|---|
| | MD direction | CD direction |
| | (mN) | (mN) |
| Sample 6 | 6292 | 4938 |
| Comparative | 282 | 238 |
| Sample | | |

The Comparative sample had a basis weight of 90 g/m² and sample 6 had a basis weight of 332 g/m² but the material according to the present disclosure had a much higher resistance to bending. Such resistance makes sample 6 suitable for making a rigid tray according to the present disclosure.

This disclosure further encompasses the following aspects.

Aspect 1. A sterilizable fibrous material for the packaging of medical devices intended to be sterilized, characterized in that the sterilizable fibrous material is in the form of a cardboard having a basis weight of at least 180 g/m², advantageously at least 200 g/m²; and the sterilizable fibrous material comprises a mixture of fibers containing at least 75% by weight of natural cellulose fibers, the length of which is less than 5 mm; and the sterilizable fibrous material has a permeability to air of at least 1.7, preferably at least 3.4, advantageously strictly greater than 3.4 μm/Pa·s at a pressure of 1.47 kPa, measured according to the ISO 5636-3 standard; and the sterilizable fibrous material has pores having a maximum pore diameter less than 50 μm, measured according to appendix C of standard EN 868-3.

Aspect 2. The material according to Aspect 1, characterized in that the mixture of fibers contains: between 20% and 100% by weight of natural long cellulose fibers, the length of which is between 1 and 5 mm; and between 0% and 80% by weight of natural short cellulose fibers, the length of which is less than 1 mm.

Aspect 3. The material according to any one of the preceding Aspects, characterized in that the long fibers have a length between 1 and 3 mm, and preferably between 1.4 and 2.5 mm, and the short fibers have a length between 0.2 and 1 mm, preferably between 0.3 and 1 mm.

Aspect 4. The material according to any one of the preceding Aspects, characterized in that the ratio of long fibers to short fibers is between 2 and 30.

Aspect 5. The material according to any one of the preceding Aspects, characterized in that the mixture of fibers further comprises between at least 1% by weight, preferably between 1 and 20% by weight, of chemical fibers having a titer between 0.3 and 10 dtex and a length between 2.5 and 20 mm.

Aspect 6. The material according to any one of the preceding Aspects, characterized in that the chemical fibers are chosen from the group comprising artificial fibers such as Lyocell and rayon, and synthetic fibers such as biopolymers like polylactic acid, polyhydroxyalcanoate, polybutylene succinates, polybutylene succinate co-adipates, polycaprolactones, polybutyrate adipate terephthalate, poly(hydroxybutyrate-co-hydroxyvalerate) or their copolymers.

Aspect 7. The material according to any one of the preceding Aspects, characterized in that the mixture of fibers is biosourced and/or recyclable and/or biodegradable.

Aspect 8. The material according to any one of the preceding Aspects, characterized in that it is biodegradable to a degree of 90%, or even 95% and meets the requirements of biodegradation of the standard EN 13432.

Aspect 9. The material according to any one of the preceding Aspects, characterized in that it has pores having a mean pore diameter less than 35 μm preferably between 10 μm and 35 μm, measured according to appendix C of standard EN 868-3.

Aspect 10. The material according to any one of the preceding Aspects, characterized in that it further comprises a wet strength agent, representing between 0.15 and 1% by dry weight with respect to the dry weight of cellulose.

Aspect 11. The material according to Aspect 10, characterized in that the wet strength agent is chosen from the group comprising polyamine epichlorhydrin (PAE), glyoxalated resins, such as glyoxalated polyamide (GPAM), formaldehyde-based resin.

Aspect 12. The material according to any one of the preceding Aspects, characterized in that it further comprises a sizing agent representing between 0.15 and 1% by dry weight in relation to the dry weight of cellulose.

Aspect 13. The material according to Aspect 12, characterized in that the sizing agent is chosen from the group comprising alkyl ketene dimer (AKD), alkenyl succinic anhydride (ASA), and rosin-based resin.

Aspect 14. The material according to any one of the preceding Aspects, characterized in that it has a COBB at 60 seconds, measured according to the standard ISO 535, of less than 20 g/m².

Aspect 15. The material according to any one of the preceding Aspects, characterized in that it complies with the standard DIN 58953-6 sections 3 and 4 in terms of a bacterial barrier.

Aspect 16. The material according to any one of the preceding Aspects, characterized in that it complies with the standard ISO 11607-1.

Aspect 17. The material according to any one of the preceding Aspects, characterized in that it has a thickness of at least 200, preferably 300 μm.

Aspect 18. The material according to any one of the preceding Aspects, characterized in that it further comprises a coating layer, the composition of which is able to render it heat-sealable

Aspect 19. The material according to any one of the preceding Aspects, characterized in that it is calendered.

Aspect 20. The material according to any one of the preceding Aspects, characterized in that the mixture of fibers further comprises between 1 and 40% by weight mercerized cellulose fibers.

Aspect 21. The material according to any one of the preceding Aspects, characterized in that the material has a resistance to bending of at least 1000 mN in a machine direction and a cross-machine direction, preferably at least 2000 mN, and even more preferably at least 3000 mN, according to ISO 2493-2, Paper and Board, Determination of resistance to bending, part 2: Taber Type tester Standard.

Aspect 22. The material according to any one of the preceding Aspects, characterized in that the material has an overall water vapor transmission rate (WVTR) of at least 400 g/m²/day, preferably at least 800 g/m²/day, and more preferably at least 1000 g/m²/day at 38° C. and 90% relative humidity and as determined by the ISO 2528 standard.

Aspect 23. Tray obtained from the material specified by any one of Aspects 1 to 22.

Aspect 24. Use of the material specified by any one of Aspects 1 to 22 for the fabrication of a tray.

Aspect 25. Packaging for medical sterilization, comprising: a tray according to Aspect 23; and a means of tight sterilizable closure of the tray.

Aspect 26. A container for the packaging of medical devices intended to be sterilized comprising: a tray comprising a sterilizable fibrous material, the tray extending from a first end to a second end, and extending from a first side to a second side, the first end including a first end wall and the second end including a second end wall, and the first side including a first side wall and the second side including a second side wall, with the first and second end walls and the first and second side walls defining a recess or cavity within internal boundaries of the first and second end walls and the first and second side walls, and with a bottom wall at a bottom of the recess or cavity; wherein the tray has a basis weight of at least 180 g/m², advantageously at least 200 g/m²; and wherein the tray comprises a mixture of fibers containing at least 75% by weight of natural cellulose fibers, the length of which is less than 5 mm; and wherein the tray has a permeability to air of at least 1.7, preferably at least 3.4, advantageously strictly greater than 3.4 μm/Pa·s at a pressure of 1.47 kPa, measured according to the ISO 5636-3 standard; and wherein the tray has pores having a maximum pore diameter less than 50 μm, measured according to appendix C of standard EN 868-3.

Aspect 27. A method for fabricating a container for the packaging of medical devices intended to be sterilized comprising: providing a sterilizable fibrous material; and forming the sterilizable fibrous material in to a tray, the tray extending from a first end to a second end, and extending from a first side to a second side, the first end including a first end wall and the second end including a second end wall, and the first side including a first side wall and the second side including a second side wall, with the first and second end walls and the first and second side walls defining a recess or cavity within internal boundaries of the first and second end walls and the first and second side walls, and with a bottom wall at a bottom of the recess or cavity; wherein the tray has a basis weight of at least 180 g/m², advantageously at least 200 g/m²; and wherein the tray comprises a mixture of fibers containing at least 75% by weight of natural cellulose fibers, the length of which is less than 5 mm; and wherein the tray has a permeability to air of at least 1.7, preferably at least 3.4, advantageously strictly greater than 3.4 μm/Pa·s at a pressure of 1.47 kPa, measured according to the ISO 5636-3 standard; and wherein the tray has pores having a maximum pore diameter less than 50 μm, measured according to appendix C of standard EN 868-3.

Aspect 28. The method of Aspect 27, wherein forming the sterilizable fibrous material in to the tray comprises thermoforming the sterilizable fibrous material or molding the sterilizable fibrous material.

While particular embodiments have been described, alternatives, modifications, variations, improvements, and substantial equivalents that are or may be presently unforeseen may arise to applicants or others skilled in the art. Accordingly, the appended claims as filed and as they may be amended are intended to embrace all such alternatives, modifications variations, improvements, and substantial equivalents.

All ranges disclosed herein are inclusive of the endpoints, and the endpoints are independently combinable with each other (e.g., ranges of “up to 25 wt. %, or, more specifically, 5 wt. % to 20 wt. %”, is inclusive of the endpoints and all intermediate values of the ranges of “5 wt. % to 25 wt. %,” etc.). “Combinations” is inclusive of blends, mixtures, alloys, reaction products, and the like. The terms “first,” “second,” and the like, do not denote any order, quantity, or importance, but rather are used to distinguish one element from another. The terms “a” and “an” and “the” do not denote a limitation of quantity and are to be construed to cover both the singular and the plural, unless otherwise indicated herein or clearly contradicted by context. “Or” means “and/or” unless clearly stated otherwise. Reference throughout the specification to “some embodiments”, “an embodiment”, and so forth, means that a particular element described in connection with the embodiment is included in at least one embodiment described herein, and may or may not be present in other embodiments. In addition, it is to be understood that the described elements may be combined in any suitable manner in the various embodiments. A “combination thereof” is open and includes any combination comprising at least one of the listed components or properties optionally together with a like or equivalent component or property not listed.

Unless defined otherwise, technical and scientific terms used herein have the same meaning as is commonly understood by one of skill in the art to which this application belongs. All cited patents, patent applications, and other references are incorporated herein by reference in their entirety. However, if a term in the present application contradicts or conflicts with a term in the incorporated reference, the term from the present application takes precedence over the conflicting term from the incorporated reference.

Unless specified to the contrary herein, all test standards are the most recent standard in effect as of the filing date of this application, or, if priority is claimed, the filing date of the earliest priority application in which the test standard appears.

Although the materials, systems and methods of the present disclosure have been described with reference to exemplary embodiments thereof, the present disclosure is not limited to such exemplary embodiments and/or implementations. Rather, the materials, systems and methods of the present disclosure are susceptible to many implementations and applications, as will be readily apparent to persons skilled in the art from the disclosure hereof. The present disclosure expressly encompasses such modifications, enhancements and/or variations of the disclosed embodiments. Since many changes could be made in the above construction and many widely different embodiments of this disclosure could be made without departing from the scope thereof, it is intended that all matter contained in the drawings and specification shall be interpreted as illustrative and not in a limiting sense. Additional modifications, changes, and substitutions are intended in the foregoing disclosure. Accordingly, it is appropriate that the appended claims be construed broadly and in a manner consistent with the scope of the disclosure.

1. A sterilizable fibrous material for the packaging of medical devices intended to be sterilized, characterized in that:
the sterilizable fibrous material is in the form of a cardboard having a basis weight of at least 180 g/m², advantageously at least 200 g/m²; and
the sterilizable fibrous material comprises a mixture of fibers containing at least 75% by weight of natural cellulose fibers, the length of which is less than 5 mm; and
the sterilizable fibrous material has a permeability to air of at least 1.7, preferably at least 3.4, advantageously strictly greater than 3.4 μm/Pa·s at a pressure of 1.47 kPa, measured according to the ISO 5636-3 standard; and
the sterilizable fibrous material has pores having a maximum pore diameter less than 50 μm, measured according to appendix C of standard EN 868-3.

2. The material according to claim 1, characterized in that the mixture of fibers contains:
between 20% and 100% by weight of natural long cellulose fibers, the length of which is between 1 and 5 mm; and
between 0% and 80% by weight of natural short cellulose fibers, the length of which is less than 1 mm.

3. The material according to claim 2, characterized in that the long cellulose fibers have a length between 1 and 3 mm, and preferably between 1.4 and 2.5 mm, and the short cellulose fibers have a length between 0.2 and 1 mm, preferably between 0.3 and 1 mm.

4. The material according to claim 2, characterized in that the ratio of long cellulose fibers to short cellulose fibers is between 2 and 30.

5. The material according to claim 2, characterized in that the mixture of fibers further comprises between at least 1% by weight, preferably between 1 and 20% by weight, of chemical fibers having a titer between 0.3 and 10 dtex and a length between 2.5 and 20 mm.

6. The material according to claim 5, characterized in that the chemical fibers are chosen from the group comprising artificial fibers such as Lyocell and rayon, and synthetic fibers such as biopolymers like polylactic acid, polyhydroxyalcanoate, polybutylene succinates, polybutylene succinate co-adipates, polycaprolactones, polybutyrate adipate terephthalate, poly(hydroxybutyrate-co-hydroxyvalerate) or their copolymers.

7. The material according to claim 2, characterized in that the mixture of fibers is biosourced and/or recyclable and/or biodegradable.

8. The material according to claim 7, characterized in that it is biodegradable to a degree of 90%, or even 95% and meets the requirements of biodegradation of the standard EN 13432.

9. The material according to claim 1, characterized in that it has the pores have a mean pore diameter less than 35 μm preferably between 10 μm and 35 μm, measured according to appendix C of standard EN 868-3.

10. The material according to claim 1, characterized in that the sterilizable fibrous material it further comprises a wet strength agent, representing between 0.15 and 1% by dry weight with respect to the dry weight of cellulose.

11. The material according to claim 10, characterized in that the wet strength agent is chosen from the group comprising polyamine epichlorhydrin (PAE), glyoxalated resins, such as glyoxalated polyamide (GPAM), formaldehyde-based resin.

12. The material according to claim 1, characterized in that the sterilizable fibrous material it further comprises a sizing agent representing between 0.15 and 1% by dry weight in relation to the dry weight of cellulose.

13. The material according to claim 12, characterized in that the sizing agent is chosen from the group comprising alkyl ketene dimer (AKD), alkenyl succinic anhydride (ASA), and rosin-based resin.

14. The material according to claim 1, characterized in that the sterilizable fibrous material it has a COBB at 60 seconds, measured according to the standard ISO 535, of less than 20 g/m².

15. The material according to claim 1, characterized in that the sterilizable fibrous material complies with the standard DIN 58953-6 sections 3 and 4 in terms of a bacterial barrier.

16. The material according to claim 1, characterized in that the sterilizable fibrous material complies with the standard ISO 11607-1.

17. The material according to claim 1, characterized in that the sterilizable fibrous material has a thickness of at least 200, preferably 300 μm.

18. The material according to claim 1, characterized in that the sterilizable fibrous material further comprises a coating layer, the composition of which is able to render it heat-scalable.

19. The material according to claim 1, characterized in that it the sterilizable fibrous material is calendered.

20. The material according to claim 2, characterized in that the mixture of fibers further comprises between 1 and 40% by weight mercerized cellulose fibers.

21. The material according to claim 1, characterized in that the sterilizable fibrous material has a resistance to bending of at least 1000 mN in a machine direction and a cross-machine direction, preferably at least 2000 mN, and even more preferably at least 3000 mN, according to ISO 2493-2, Paper and Board, Determination of resistance to bending, part 2: Taber Type tester Standard.

22. The material according to any one of the preceding claims, characterized in that the sterilizable fibrous material has an overall water vapor transmission rate (WVTR) of at least 400 g/m²/day, preferably at least 800 g/m²/day, and more preferably at least 1000 g/m²/day at 38° C. and 90% relative humidity and as determined by the ISO 2528 standard.

23. Tray obtained from the sterilizable fibrous material specified by claim 1.

24. Use of the material specified by claim 1 for the fabrication of a tray.

25. Packaging for medical sterilization, comprising:
a tray according to claim 23; and
a means of tight sterilizable closure of the tray.

26. A container for the packaging of medical devices intended to be sterilized comprising:
a tray comprising a sterilizable fibrous material, the tray extending from a first end to a second end, and extending from a first side to a second side, the first end including a first end wall and the second end including a second end wall, and the first side including a first side wall and the second side including a second side wall, with the first and second end walls and the first and second side walls defining a recess or cavity within internal boundaries of the first and second end walls and the first and second side walls, and with a bottom wall at a bottom of the recess or cavity;
wherein the tray has a basis weight of at least 180 g/m², advantageously at least 200 g/m²; and
wherein the tray comprises a mixture of fibers containing at least 75% by weight of natural cellulose fibers, the length of which is less than 5 mm; and
wherein the tray has a permeability to air of at least 1.7, preferably at least 3.4, advantageously strictly greater than 3.4 μm/Pa·s at a pressure of 1.47 kPa, measured according to the ISO 5636-3 standard; and
wherein the tray has pores having a maximum pore diameter less than 50 μm, measured according to appendix C of standard EN 868-3.

27. A method for fabricating a container for the packaging of medical devices intended to be sterilized comprising:
providing a sterilizable fibrous material; and
forming the sterilizable fibrous material in to a tray, the tray extending from a first end to a second end, and extending from a first side to a second side, the first end including a first end wall and the second end including a second end wall, and the first side including a first side wall and the second side including a second side wall, with the first and second end walls and the first and second side walls defining a recess or cavity within internal boundaries of the first and second end walls and the first and second side walls, and with a bottom wall at a bottom of the recess or cavity;
wherein the tray has a basis weight of at least 180 g/m², advantageously at least 200 g/m²; and
wherein the tray comprises a mixture of fibers containing at least 75% by weight of natural cellulose fibers, the length of which is less than 5 mm; and
wherein the tray has a permeability to air of at least 1.7, preferably at least 3.4, advantageously strictly greater than 3.4 μm/Pa·s at a pressure of 1.47 kPa, measured according to the ISO 5636-3 standard; and
wherein the tray has pores having a maximum pore diameter less than 50 μm, measured according to appendix C of standard EN 868-3.

28. The method of claim 27, wherein forming the sterilizable fibrous material in to the tray comprises thermoforming the sterilizable fibrous material or molding the sterilizable fibrous material.
